(19) United States
(12) Patent Application Publication
(10) Pub. No.: US 2007/0032401 A1
(43) Pub. Date: Feb. 8, 2007

(54) Macrocyclic musks

(75) Inventor: Luca Turin, London (GB)

Correspondence Address: MILES & STOCKBRIDGE PC, 1751 PINNACLE DRIVE, SUITE 500, MCLEAN, VA, 22102-3833, US

(73) Assignee: FLEXITRAL, INC., Chantilly, VA (US)

(21) Appl. No.: 10/548,471

(22) PCT Filed: Mar. 12, 2004
(86) PCT No.: PCT/US04/07977

§ 371 Date: Jun. 1, 2006

Related U.S. Application Data
(60) Provisional application No. 60/453,969, filed on Mar. 13, 2003.

(51) Int. Cl. A61K 8/00

(52) U.S. Cl. 512/3

(57) ABSTRACT

A compound comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of said oxiranes or thiiranes containing a methyl substituent on the ring and/or an ether oxygen or thioether sulfur substituting for a CH₂ somewhere in the ring and compositions, products, preparations or articles having improved aroma, fragrance, odor, flavor and/or taste characteristics containing as active ingredient a compound or mixture of the compounds.

### FIELD OF THE INVENTION

The present invention relates generally to the field of fragrances. More particularly, the present invention relates to improved derivatives that provide perfumes and other articles with a musk like odor. These derivatives find utility in applications requiring the themes supplied by the macrocyclic musks. The invention relates to novel macrocyclic musks and mixtures of macrocyclic musks and their use as perfume materials for application to a variety of substrates.

### DESCRIPTION OF THE PRIOR ART

Musk fragrances are well known and much used perfume ingredients in perfumes for a large range of products. For example, perfumes for application in laundry detergents, fabric softeners, rinse conditioners and other products intended for use on textile fibers primarily contain musk fragrances of the class of macrocyclic and polycyclic musks. Musk fragrances are present in many perfume oils in not inconsiderable amounts. Accordingly, the annual worldwide requirement for musk fragrances is several thousand tons. By far the largest part is provided by the so-called polycyclic aromatic musk fragrances. It has become known that polycyclic aromatic musk fragrances (well known examples of this class are marketed under various tradenames such as Extralide, Tonalide, Traseolide, Galaxolide, etc.) can only be biodegraded with difficulty and consequently, being extremely lipophilic compounds, exhibit bioaccumulative behavior, i.e. they are able to accumulate in the fatty tissue of organisms. In the perfume industry, there is therefore, a pressing need for biodegradable musk fragrances which are suitable both in terms of the odiferous properties and also in terms of price as replacements for the polycyclic aromatic compounds. In contrast to the polycyclic aromatic compounds, macrocyclic musk fragrances are regarded as biodegradable (U.S. Pat. No. 6,034,052). Typical such musk fragrances are characterized by a macrocyclic ring having 13 to 17 carbon atoms which carries a ketone or an ester as functional group. Moreover, macrocyclic musk fragrances which carry two functional groups are also known, e.g. 1,7-dioxacycloalkan-8-ones (EP A 884,315).

Macrocyclic musks are also long known in perfumery; well known examples of this class of perfume ingredients are the macrocyclic ketones and macrocyclic lactones (macrolides) or dilactones, e.g., cyclohexadecanone, hexadecanolide, cyclopentadecanone, cyclopentadecanolide and various unsaturated and/or methyl substituted analogues thereof. [Williams, Synthesis 1999, No. 10, 1707-1723]

Improved fragrances and flavorings that have odorant intensities similar to the macrocyclic musks are disclosed. In particular, derivatives that maintain or improve the flavor and/or fragrance characteristics of the macrocyclic musks are disclosed. Also disclosed are methods of making the derivatives, and articles of manufacture including the derivatives.

### SUMMARY OF THE INVENTION

The invention is directed toward certain macrocyclic musks containing at least one double bond, wherein at least one double bond has been converted to an oxirane or thiirane. For example, the double bond of the well known macrocyclic musks having the formulae: 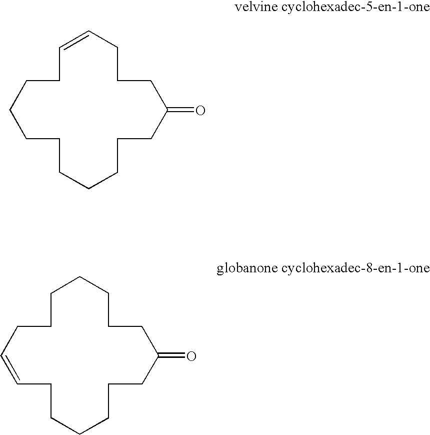
is converted according to the invention to an oxirane group and then, if desired, to a thiirane group according to the following scheme:

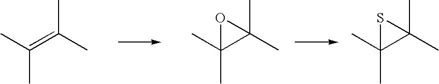

One embodiment of the invention relates to a compound or mixtures of compounds comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of the oxiranes or thiiranes containing a methyl substituent on the ring and/or an ether oxygen or thioether sulfur substituting for a CH₂ somewhere in the ring; excluding the compounds having the formula:

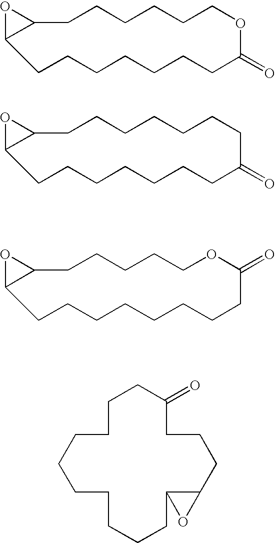

A second embodiment of the invention concerns compositions, products, preparations or articles having improved aroma, fragrance or odor characteristics containing as active ingredient a compound or mixture of compounds comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of the oxiranes or thiiranes containing a methyl substituent on the ring and/or an ether oxygen or thioether sulfur substituting for a CH₂ somewhere in the ring.

Another embodiment of the invention relates to compositions, products, preparations or articles having improved flavor or taste characteristics containing as active ingredient a compound or mixtures of compounds comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of the oxiranes or thiiranes containing a methyl substituent on the ring and/or an ether oxygen or thioether sulfur substituting for a CH₂ somewhere in the ring.

A further embodiment of the invention concerns methods to confer, improve, enhance or modify the taste or flavor property of a composition, product, preparation or article which comprises adding thereto a flavor effective amount of a compound or mixtures of compounds comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of the oxiranes or thiiranes containing a methyl substituent on the ring and/or an ether oxygen or thioether sulfur substituting for a CH₂ somewhere in the ring.

A still further embodiment of the invention relates to a method to confer, improve, enhance or modify the aroma, fragrance or odor characteristics of compositions, products, preparations or articles which comprises adding thereto an aroma, fragrance or odor effective amount of a compound or mixture of compounds comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of the oxiranes or thiiranes containing a methyl substituent on the ring and/or an ether oxygen or thioether sulfur substituting for a CH₂ somewhere in the ring.

Examples of suitable articles of manufacture in which the derivatives of the invention may be incorporated include perfumes and colognes, candles, air fresheners, detergent compositions and disinfectant compositions.

The derivatives of the invention can be used, for example, as fragrances in any applications in which the corresponding macrocyclic musks are applied.

### DETAILED DESCRIPTION OF THE INVENTION

The present invention is predicated on the discovery of improved fragrances and flavorings that have increased odorant and/or flavorant intensity relative to conventional macrocyclic musks. In particular, the compounds of the invention maintain the flavor and/or fragrance characteristics of the analogous macrocyclic musks, while increasing the odor intensity relative thereto. Also disclosed are methods of making the compounds, utilizing the derivatives as flavorants and/or fragrances and compositions, products, preparations and articles incorporating the derivatives. The derivatives of the invention find utility in any application requiring musk themes. The compositions, products, preparations and articles in which the compounds and derivatives of the invention may be incorporated include candles, air fresheners, perfumes, fragrances, colognes, soaps, bath or shower gels, shampoos or other hair care products, cosmetic preparations, body odorants, deodorants or antiperspirants, liquid or solid fabric detergents or softeners, bleach products (hypochlorites), disinfectants, all-purpose household or industrial cleaners, foods, flavorings, beverages such as beer and soda, denture cleansers (tablets), flavored orally-delivered products such as lozenges, candies, chewing gums, matrices, pharmaceuticals and the like.

The derivatives of the invention can be viewed as macrocyclic musks thereof wherein the double bond therein has been replaced by an oxirane or thiirane group. More specifically, the novel derivatives of the invention comprise macrocyclic ketones having at least one double bond and 15-20 carbon atoms, wherein at least one double bond has been converted to oxirane or thiirane or macrocyclic lactones (macrolides) or dilactones having at least one double bond and 14-19 carbon atoms, wherein at least one double bond has been converted to oxirane or thiirane; excluding the compounds having the formulas: 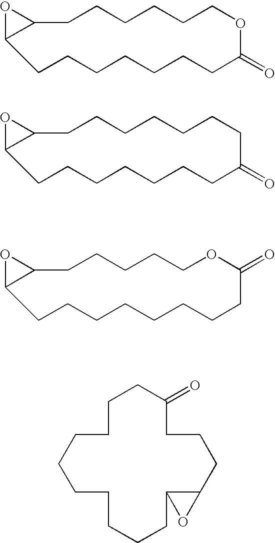
which are known in the art; however, not for use as perfume or odorant materials.

The derivatives of the invention also include all of the above described oxiranes and thiiranes containing a methyl substituent on the ring and/or an ether oxygen or thioether sulfur substituting for a CH₂ somewhere in the ring. The derivatives of the invention can be prepared according to the methods described in Bouda, N., et al (1989) Synthesis of Epoxides from Thiiranes in the presence of Thiourea. Synthetic Communications 199:491-500.

### EXAMPLE 1

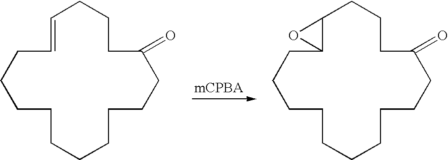

In a 500-mL round bottom flask containing velvione (5-Cyclohexadecen-1-one, 5 g) in dichloromethane (200 mL) was added mCPBA (8.0 g, 50-85%) portionwise over 45 minutes and the solution stirred for 5 hours. The mixture was washed with a solution of saturated Na₂SO₃ (70 mL), followed by washing with brine (3×70 mL). The organic phase was dried over Na₂SO₄, filtered and the solvent evaporated in vacuo to give the pure desired product as a colorless oil (6.0 g )

### EXAMPLE 2

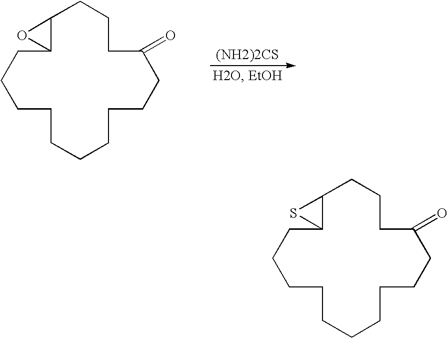

To the epoxide product from example 1 in ethanol (10 mL) and water (6 mL) was added thiourea (1.6 g) and the solution stirred overnight under a N₂ atmosphere. The solvent was evaporated in vacuo. Flash column chromatography from the mixture using dichloromethane/ethyl acetate (3:2) afforded the desired thiirane as colorless oil.

The derivatives of the invention can be included in virtually any article of manufacture that can include conventional macrocyclic musks, or for that matter, other fragrances, whether natural or artificial. Examples include bleach, detergents, flavorings and fragrances, beverages, including alcoholic beverages, and the like. The macrocyclic musk derivatives can be used in applications like soaps, shampoos, body deodorants and antiperspirants, solid or liquid detergents for treating textiles, fabric softeners, detergent compositions and/or all-purpose cleaners for cleaning dishes or various surfaces, for both household and industrial use. Of course, the use of the compounds is not limited to the above-mentioned products, as they be used in other current uses in perfumery, namely the perfuming of soaps and shower gels, hygiene or hair-care products, as well as of body deodorants, air fresheners and cosmetic preparations, and even in fine perfumery, namely in perfumes and colognes. The products of the invention also find utility in foods, flavorings, beverages such as beer and soda, denture cleansers (tablets), flavored orally-delivered products such as lozenges, candies, chewing gums, matrices, pharmaceuticals and the like. These uses are described in more detail below.

The compounds can be used as perfuming ingredients, as single compounds or as mixture thereof, preferably at a range of at least about 30% by weight of the perfume composition, more preferably at a range of at least about 60% by weight of the composition. The compounds can even be used in their pure state or as mixtures, without added components. The olfactive characteristics of the individual compounds are also present in mixtures thereof, and mixtures of these compounds can be used as perfuming ingredients. This may be particularly advantageous where separation and/or purification steps can be avoided by using compound mixtures.

In all cited applications, the macrocyclic musk derivatives can be used alone, in admixture with each other, or in admixture with other perfuming ingredients, solvents or adjuvants of current use in the art. The nature and the variety of these co-ingredients do not require a more detailed description here, which, moreover, would not be exhaustive, and the person skilled in the art will be able to choose the latter through its general knowledge and as a function of the nature of the product to be perfumed and of the desired olfactive effect.

These perfuming ingredients typically belong to chemical classes as varied as alcohols, aldehydes, ketones, esters, ethers, acetates, nitrites, terpene hydrocarbons, sulfur- and nitrogen containing heterocyclic compounds, as well as essential oils of natural or synthetic origin. A large number of these ingredients described in reference textbooks such as the book of S. Arctander, Perfume and Flavor Chemicals, 1969, Montclair, N.J., USA, the contents of which are hereby incorporated by reference in its entirety, or its more recent versions, or in other works of similar nature.

The proportions in which the macrocyclic musk derivatives can be incorporated in the various products vary within a large range of values. These values depend on the nature of the article or product that one desires to perfume and the odor effect searched for, as well as on the nature of the co-ingredients in a given composition when the compounds are used in admixture with perfuming co-ingredients, solvents or adjuvants of current use in the art.

As an example, the macrocyclic musk derivatives are typically present at concentrations between about 0.1 and about 10%, or even more, by weight of these compounds relative to the weight of the perfuming composition in which they are incorporated. Far lower concentrations than those mentioned above can be used when the compounds are directly applied for perfuming the various consumer products cited beforehand.

The compounds may be used in detergents containing bleaching agents and activators such as, for example, tetraacetylethylenediamine (TAED), hypohalites, in particular hypochlorite, peroxygenated bleaching agents such as, for example, perborates, etc. The compounds can also be used in body deodorants and antiperspirants, for example, those containing aluminum salts. These embodiments are described in more detail below.

In addition to the derivatives described herein, the compositions herein include a detersive surfactant and optionally, one or more additional detergent ingredients, including materials for assisting or enhancing cleaning performance, treatment of the substrate to be cleaned, or to modify the aesthetics of the detergent composition (e.g., perfumes, colorants, dyes, etc.). Non-limiting examples of synthetic detersive surfactants useful herein typically at levels from about 0.5% to about 90%, by weight, include the conventional C1-18 alkyl benzene sulfonates (“LAS”) and primary, branch-chain and random CIO-20 alkyl sulfates (“AS”), and the like.

Preferred compositions incorporating only synthetic detergents have a detergent level of from about 0.5% to 50%. Compositions containing soap preferably comprise from about 10% to about 90% soap.

The compositions herein can contain other ingredients such as enzymes, bleaches, fabric softening agents, dye transfer inhibitors, suds suppressors, and chelating agents, all well known within the art.

The macrocyclic musk derivatives described herein can be incorporated into beverages and impart various flavorings to the beverages. The beverage composition can be a cola beverage composition, and can also be coffee, tea, dairy beverage, fruit juice drink, orange drink, lemon-lime drink, beer, malt beverages, or other flavored beverage. The beverages can be in liquid or powdered form. The beverage compositions can also include one or more flavoring agents; artificial colorants; vitamin additives; preservatives; caffeine additives; water; acidulants; thickeners; buffering agents; emulsifiers; and or fruit juice concentrates.

Artificial colorants which may be used include caramel color, yellow 6 and yellow 5. Useful vitamin additives include vitamin B2, vitamin B6, vitamin B12, vitamin C (ascorbic acid), niacin, pantothenic acid, biotin and folic acid. Suitable preservatives include sodium or potassium benzoate. Salts which may be used include sodium, potassium and magnesium chloride. Exemplary emulsifiers are gum arabic and purity gum, and a useful thickener is pectin. Suitable acidulants include citric, phosphoric and malic acid, and potential buffering agents include sodium and potassium citrate.

In one embodiment, the beverage is a carbonated cola beverage. The pH is generally about 2.8 and the following ingredients can be used to make the syrup for these compositions: Flavor Concentrate, including one or more of the derivatives described herein (22.22 ml), 80% Phosphoric Acid (5.55 g), Citric Acid (0.267 g), Caffeine (1.24 g), artificial sweetener, sugar or corn syrup (to taste, depending on the actual sweetener) and Potassium Citrate (4.07 g). The beverage composition can be prepared, for example, by mixing the foregoing syrup with carbonated water in a proportion of 50 ml syrup to 250 ml of carbonated water.

Flavored food and pharmaceutical compositions including one or more of the derivatives described herein can also be prepared. The derivatives can be incorporated into conventional foodstuffs using techniques well known to those of skill in the art. Alternatively, the derivatives can be incorporated within polymeric particles, which can, in turn, be dispersed within and/or over a surface of an orally-deliverable matrix material, which is usually a solid or semi-solid substrate. When used in chewable compositions, the derivatives can be released into the orally-deliverable polymeric matrix material as the composition is chewed and held in the mouth, thus prolonging the flavor of the composition. In the case of dried powders and mixes, the flavor can be made available as the product is consumed or be released into the matrix material as the composition is further processed. When two flavors are combined with the polymeric particles, the relative amounts of the additives can be selected to provide simultaneous release and exhaustion of the compounds.

In one embodiment, the flavored composition includes an orally-deliverable matrix material; a plurality of water insoluble polymeric particles dispersed in the orally-deliverable matrix material, where the polymeric particles individually define networks of internal pores and are non-degradable in the digestive tract; and one or more derivatives as described herein entrapped within the internal pore networks. The derivatives are released as the matrix is chewed, dissolved in the mouth, or undergoes further processing selected from the group consisting of liquid addition, dry blending, stirring, mixing, heating, baking, and cooking. The orally-deliverable matrix material can be selected from the group consisting of gums, latex materials, crystallized sugars, amorphous sugars, fondants, nougats, jams, jellies, pastes, powders, dry blends, dehydrated food mixes, baked goods, batters, doughs, tablets, and lozenges.

A flavorless gum base can be combined with a macrocyclic musk derivative or other suitable derivative as described herein to a desired flavor concentration. Typically, a blade mixer is heated to about 11 OF, the gum base is preheated so that it is softened, and the gum base is then added to the mixer and allowed to mix for approximately 30 seconds. The flavored derivative is then added to the mixer and mixed for a suitable amount of time. The gum can be then removed from the mixer and rolled to stick thickness on waxed paper while warm.

In one embodiment, the derivatives described herein are incorporated into a system which can release a fragrance in a controlled manner. These include substrates such as air fresheners, laundry detergents, fabric softeners, deodorants, lotions, and other household items. The fragrances are generally one or more derivatives of essential oils as described herein, each present in different quantities. U.S. Pat. No. 4,587,129, the contents of which are hereby incorporated by reference in their entirety, describes a method for preparing gel articles which contain up to 90% by weight of fragrance or perfume oils. The gels are prepared from a polymer having a hydroxy (lower alkoxy) 2-alkeneoate, a hydroxy (lower alkoxy) lower alkyl 2-alkeneoate, or a hydroxy poly (lower alkoxy)lower alkyl 2-alkeneoate and a polyethylenically unsaturated crosslinking agent. These materials have continuous slow release properties, i.e., they release the fragrance component continuously over a long period of time. Advantageously, all or a portion of those derivatives that include an aldehyde group can be modified to include an acetal group, which can cause the formulations to release fragrance over a period of time as the acetal hydrolyzes to form the aldehyde compound.

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

I claim:

1. A compound or a mixture of compounds comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of said oxiranes or thiiranes containing a methyl substituent on the ring and/or an ether oxygen or thioether sulfur substituting for a CH₂ somewhere in the ring; excluding the compounds having the formula:

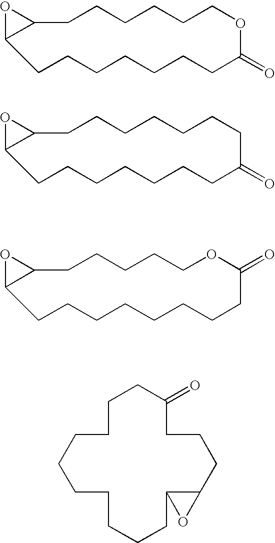

2. A composition, product, preparation or article having improved aroma, fragrance or odor characteristics containing as active ingredient a compound or mixture of compounds comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of said oxiranes or thiiranes containing a methyl substituent on the ring and/or an ether oxygen or thioether sulfur substituting for a CH₂ somewhere in the ring.

3. The composition, product, preparation or article of claim 2, wherein the compound is present in an amount of at least 30 percent by weight.

4. The composition, product, preparation or article of claim 2, wherein the compound is present in an amount of at least 60 percent by weight.

5. A composition, product, preparation or article of claim 3 in the form of a perfume, fragrance or cologne, a soap, a bath or shower gel, a shampoo or other hair care product, a cosmetic preparation, a body odorant, deodorant or antiperspirant, an air freshener, a liquid or solid fabric detergent or softener, bleach product, disinfectant or an all-purpose household or industrial cleaner.

6. A perfuming composition, product, preparation or article of claim 5, wherein the compound or mixture of compounds is present in admixture with other perfuming ingredients, solvents, or adjuvants of current use in the art.

7. A composition, product, preparation or article of claim 6 in the form of a body odorant, deodorant or antiperspirant wherein the compound or mixture of compounds is in admixture with other body odorant, deodorant or antiperspirant ingredients, solvents or adjuvants.

8. A perfumed article according to claim 5, in the form of a perfume or cologne, a soap, a bath or shower gel, a shampoo or other hair care product, a cosmetic preparation, a body deodorant or antiperspirant, an air freshener, a fabric detergent or softener or an all-purpose household cleaner.

9. A composition, product, preparation or article of claim 8 in the form of a perfume wherein the compound or mixture of compounds is in admixture with other perfuming ingredients, solvents or adjuvants.

10. A body deodorant or antiperspirant composition, product, preparation or article, of claim 5.

11. A body deodorant or antiperspirant composition, product, preparation or article, of claim 10, wherein the compound or mixture of compounds is present in admixture with other perfuming ingredients, solvents, or adjuvants of current use in the art.

12. A detergent composition, product, preparation or article of claim 5.

13. A detergent composition, product, preparation or article of claim 12 wherein the compound or mixture of compounds is in admixture with other detergent ingredients, solvents or adjuvants.

14. A bleach composition, product, preparation or article of claim 5.

15. A bleach composition, product, preparation or article of claim 13 wherein the compound or mixture of compounds is in admixture with other bleach ingredients, solvents or adjuvants.

16. A composition, product, preparation or article of claim 5 in the form of a disinfectant.

17. The disinfectant composition, product, preparation or article of claim 16 wherein the compound or mixture of compounds is in admixture with other disinfectant ingredients, solvents or adjuvants.

18. A composition, product, preparation or article having improved flavor or taste characteristics containing as active ingredient a compound or mixture of compounds comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of said oxiranes or thiiranes containing a methyl substituent on the ring and/or an ether oxygen or thioether sulfur substituting for a CH₂ somewhere in the ring.

19. A composition, product, preparation or article of claim 18 in the form of a beverage.

20. A beverage composition, product, preparation or article of claim 19 wherein the compound or mixture of compounds is in admixture with other beverage ingredients, solvents or adjuvants.

21. A composition, product, preparation or article of claim 18 in the form of a flavoring.

22. A flavoring composition, product, preparation or article of claim 21 wherein the compound or mixture of compounds is in admixture with other flavoring ingredients, solvents or adjuvants.

23. A composition, product, preparation or article of claim 18 in the form of a food wherein the compound or mixture of compounds is in admixture with other food ingredients, solvents or adjuvants.

24. A food composition, product, preparation or article of claim 23 wherein the compound or mixture of compounds is in admixture with other food ingredients, solvents or adjuvants.

25. A composition, product, preparation or article of claim 18 in the form of a chewing gum.

26. A chewing gum composition, product, preparation or article of claim 25 wherein the compound or mixture of compounds is in admixture with other chewing gum ingredients, solvents or adjuvants.

27. A composition, product, preparation or article of claim 18 in the form of a pharmaceutical.

28. A pharmaceutical composition, product, preparation or article of claim 27 wherein the compound or mixture of compounds is in admixture with other pharmaceutical ingredients, solvents or adjuvants.

29. A composition, product, preparation or article of claim 18 in the form of an orally-deliverable matrix material.

30. A composition, product, preparation or article of claim 29 wherein the compound or mixture of compounds is in admixture with other matrix material ingredients, solvents or adjuvants.

31. A method to confer, improve, enhance or modify the taste or flavor property of a composition, product, preparation or article which comprises adding thereto a flavor effective amount of a compound or mixture of compounds comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of said oxiranes or thiiranes containing a methyl substituent on the ring and/or an ether oxygen or thioether sulfur substituting for a CH₂ somewhere in the ring.

32. The method of claim 31 wherein said composition, product, preparation or article is in the form of a beverage.

33. The method of claim 31 wherein said composition, product, preparation or article is in the form of a flavoring.

34. The method of claim 31 wherein said composition, product, preparation or article is in the form of a food.

35. The method of claim 31 wherein said composition, product, preparation or article is in the form of a chewing gum.

36. The method of claim 31 wherein said composition, product, preparation or article is in the form of a pharmaceutical.

37. The method of claim 31 wherein said composition, product, preparation or article is in the form of an orally deliverable matrix.

38. A method to confer, improve, enhance or modify the aroma, fragrance or odor characteristics of a composition, product, preparation or article which comprises adding thereto an aroma, fragrance or odor effective amount of a compound or mixture of compounds comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of said oxiranes or thiiranes containing a methyl substituent on the ring and/or an ether oxygen or thioether sulfur substituting for a CH₂ somewhere in the ring.

39. The method of claim 38 wherein said composition, product, preparation or article is in the form of a perfume.

40. The method of claim 38 wherein said composition, product, preparation or article is in the form of a body odorant, deodorant or antiperspirant.

41. The method of claim 38 wherein said composition, product, preparation or article is in the form of a detergent.

42. The method of claim 38 wherein said composition, product, preparation or article is in the form of a bleach product.

43. The method of claim 38 wherein said composition, product, preparation or article is in the form of a disinfectant.

44. An article of manufacture comprising packaging material and an aroma, odor, fragrance, taste or flavor enhancing agent contained within said packaging material, wherein said agent is effective for the enhancement of the aroma, odor, fragrance, taste or flavor of a composition, preparation, product or article to which it is added, and wherein said packaging material comprises a label which indicates that said agent can be used for enhancing aroma, odor, fragrance, taste or flavor, and wherein said agent is a compound or mixture of compounds comprising a macrocyclic ketone having at least one double bond and 15-20 carbon atoms, wherein at least one double bond has been converted to oxirane or thiirane or a macrocyclic lactone or dilactone having at least one double bond and 14-19 carbon atoms, wherein at least one double bond has been converted to oxirane or thiirane; or one of said oxiranes or thiiranes containing a methyl substituent on the ring and/or an ether oxygen or thioether sulfur substituting for a CH₂ somewhere in the ring.
